(19) United States
(12) Patent Application Publication
(10) Pub. No.: US 2026/0199373 A1
(43) Pub. Date: Jul. 16, 2026

(54) Ergosterol-loaded brain-targeting nano-formulation, preparation method and use thereof

(71) Applicants: Wuxi No.2 People’s Hospital, Wuxi (CN); Jiangnan University, Wuxi (CN)

(72) Inventors: Mingzhu GAO, Wuxi City (CN); Xiaojie LU, Wuxi City (CN); Wentao SHI, Wuxi City (CN); Yang YE, Wuxi City (CN); Qi CHEN, Wuxi City (CN); Yujie ZHOU, Wuxi City (CN)

(21) Appl. No.: 19/428,097

(22) Filed: Dec. 20, 2025

(30) Foreign Application Priority Data
Jan. 10, 2025 (CN) 2025100433487

(51) Int. Cl. A61K 31/575; A61K 9/51; A61P 9/10

(52) U.S. Cl. CPC A61K 31/575; A61K 9/5146; A61K 9/5192; A61P 9/10

(57) ABSTRACT

The present disclosure provides an ergosterol-loaded brain-targeting nano-formulation, a preparation method thereof, in the nano-formulation, the active ingredient ergosterol is encapsulated in a DSPE-PEG-pinacol boronate nanomaterial. This nanomaterial possesses reactive oxygen species (ROS)-responsive properties. The ergosterol-loaded nano-formulation exhibits a regular spherical morphology, uniform particle size, good dispersibility, and good stability. The nano-formulation possesses a good capability for the targeted therapy of ischemic stroke. The nano-formulation effectively addresses problems associated with conventional drugs, such as poor targeting and low bioavailability. The nano-formulation enhances the therapeutic effect of ergosterol on ischemic stroke, can effectively ameliorate brain damage caused by ischemic stroke, and exhibits a good therapeutic effect. Meanwhile, the active ingredient ergosterol exerts its therapeutic effect on ischemic stroke by activating the phosphatidylinositol 3-kinase (PI3K)/protein kinase B (AKT)/mammalian target of rapamycin (mTOR) pathway, thereby providing a new direction for the treatment of ischemic stroke.

### CROSS-REFERENCE TO RELATED APPLICATIONS

This application claims priority to Chinese Patent Application No. 202510043348.7, filed on Jan. 10, 2025, which is incorporated herein by reference in its entirety.

### TECHNICAL FIELD

The present disclosure relates to the fields of nanomaterials and nanobiomedicine, and specifically relates to an ergosterol-loaded brain-targeting nano-formulation, a preparation method and use thereof.

### BACKGROUND

Stroke is an acute disease caused by vascular disorders that supply blood to the brain. It is characterized by high incidence, high disability rate, high recurrence rate, and high mortality, making it one of the most important fatal diseases worldwide. Stroke is mainly classified into ischemic stroke (IS) and hemorrhagic stroke. Ischemic stroke accounts for approximately 75% to 85% of the total number of stroke patients, severely affecting patients'quality of life.

The pathological state of IS refers to the interruption of blood flow to a part of the brain. Mitochondrial energy metabolism then shifts to anaerobic glycolysis, resulting in reduced ATP generation. The interruption of oxygen and glucose supply to cells in the ischemic core leads to a massive release of the excitatory amino acid glutamate in the central nervous system. Glutamate accumulates in the synaptic cleft, exerting excitotoxicity on neuronal cells and triggering intracellular calcium overload, which causes mitochondrial dysfunction and leads to the generation of excessive free radicals such as reactive oxygen species (ROS). Excessive ROS damages protein molecules within cells, inducing autophagy and apoptosis of the cells and causing further tissue damage.

The treatment of IS primarily includes surgical treatment and drug treatment, but is confronted with the following challenges: (1) Surgical treatment: This method restores blood perfusion to the ischemic area of the brain through surgery. However, it has a strict time window limitation, requiring surgery to be performed within a short period after onset. Otherwise, the optimal treatment opportunity may be missed, leading to poor surgical outcomes or even making surgery impossible. The risks are high, as the surgical procedure itself may cause additional damage to brain tissue, increasing patient suffering and the difficulty of postoperative recovery. Prognosis is uncertain; even if surgery is successful, the patient's prognosis is difficult to predict accurately, and varying degrees of neurological dysfunction may still remain. (2) Drug treatment: 1 Poor drug targeting: Conventional drugs have difficulty precisely reaching the lesion site in the brain when treating ischemic stroke, limiting the therapeutic effect. 2 Low bioavailability: During the absorption, distribution, metabolism, and excretion of drugs in the body, the drugs cannot fully exert their pharmacological effects. This results in drug wastage and may also necessitate increased drug dosage, thereby increasing the risk of adverse reactions. (3) Lack of effective therapeutic drugs: Clinically, therapeutic drugs for ischemic stroke are limited. A current hotspot in drug treatment is administering thrombolytic drugs for rapid thrombolysis to restore blood perfusion to the ischemic area of the brain. However, tissue-type plasminogen activator (t-PA), currently the only drug approved by the FDA (U.S. Food and Drug Administration) for acute phase treatment, has a short optimal treatment time window (≤4.5 hours) and is prone to complications such as reperfusion injury and cerebral hemorrhage. Therefore, there is an urgent need for new therapeutic drugs and methods.

### SUMMARY

To resolve the above-mentioned technical problems, the present disclosure provides an ergosterol-loaded brain-targeting nano-formulation, a preparation method and use thereof. Through bioinformatics analysis and in vivo and in vitro validation experiments, it was discovered that ergosterol can prevent and treat IS by activating the phosphatidylinositol 3-kinase (PI3K)/protein kinase B (AKT)/mammalian target of rapamycin (mTOR) signaling pathway. Combining this discovery with the pathological state of IS, a ROS-responsive nanomaterial, DSPE-PEG-pinacol boronate, was synthesized. Ergosterol was encapsulated using this nanomaterial to prepare an ergosterol nano-formulation, achieving targeted therapy for IS. This nano-formulation can effectively address problems such as poor drug targeting and low bioavailability, opens up new treatment methods for ischemic stroke, and enhances the targeted therapeutic capability of the drug. Meanwhile, it was discovered that ergosterol exerts its therapeutic effect on ischemic stroke by activating the PI3K/AKT/mTOR pathway.

To achieve the above objective, the present disclosure first provides an ergosterol-loaded brain-targeting nano-formulation, wherein the nano-formulation includes ergosterol, pinacol boronate and DSPE-PEG; wherein the pinacol boronate modifies DSPE-PEG to form a DSPE-PEG-pinacol boronate nanomaterial for encapsulating the ergosterol.

Preferably, a zeta potential of the nano-formulation is −23.88±0.38 mV, a particle size of the nano-formulation is 30 nm, a drug loading capacity of the nano-formulation is 8.05% to 18.33%, and an encapsulation efficiency of the nano-formulation is 85.20% to 93.77%.

Based on a general inventive concept, this solution further provides a method for preparing the nano-formulation, including the following steps:
S1, activating pinacol boronate: mixing 4-(hydroxymethyl)phenylboronic acid pinacol ester with N,N′-carbonyldiimidazole, adding anhydrous dichloromethane, and stirring to react; after the reaction is completed, extracting with anhydrous dichloromethane, washing three times with deionized water, further extracting the resulting organic phase with a saturated sodium chloride solution, and drying over anhydrous sodium sulfate to obtain the activated pinacol boronate; and
S2, preparing the DSPE-PEG-pinacol boronate nanomaterial: dissolving the activated pinacol boronate obtained in S1, β-cyclodextrin, 4-dimethylaminopyridine, and DSPE-PEG together in dimethyl sulfoxide to react; adding anhydrous diethyl ether to the reaction solution for precipitation, washing with anhydrous diethyl ether, centrifuging to collect a precipitate, and performing freeze-drying under a vacuum environment to obtain the DSPE-PEG-pinacol boronate nanomaterial; and
S3, preparing the nano-formulation: placing ergosterol and the DSPE-PEG-pinacol boronate nanomaterial obtained in S2 in a round-bottom flask; adding an organic solvent; stirring until complete dissolution; connecting to a rotary evaporator for slow rotary evaporation until the organic solvent is completely evaporated and a lipid film is formed on an inner wall of the round-bottom flask; adding a phosphate-buffered saline (PBS) buffer solution; stirring in a 37° C. water bath to allow the lipid film to fully hydrate to form a suspension; performing ultrasonication and dialysis purification on the suspension; and freeze-drying to obtain the nano-formulation.

Preferably, in step S1, the mass ratio of the 4-(hydroxymethyl)phenylboronic acid pinacol ester to the N,N′-carbonyldiimidazole is (1-2):(1-2.5), and the stirring time is 1 h.

Preferably, in step S2, the mass ratio of the activated pinacol boronate to the β-cyclodextrin to the 4-dimethylaminopyridine is (1-2):(0.1-1):(0.5-1).

Preferably, in step S3, the mass ratio of ergosterol to the DSPE-PEG-pinacol boronate nanomaterial is 1: (4-12).

Preferably, in step S3, the organic solvent is selected from any one or more of methanol, dichloromethane, and chloroform.

Preferably, in step S3, a temperature for the slow rotary evaporation is from room temperature to 45° C., and a pressure for the slow rotary evaporation is 0.05-0.15 MPa.

Based on a general inventive concept, this solution further provides a use of the ergosterol-loaded brain-targeting nano-formulation for targeted therapy of a brain disease.

Preferably, the brain disease includes ischemic stroke.

The therapeutic mechanism of the nano-formulation of this solution is as follows:

This solution is based on the first discovery that ergosterol can prevent and treat ischemic stroke by activating the PI3K/AKT/mTOR signaling pathway, and the nano-formulation was thus constructed based on this discovery. When ischemic stroke occurs, the ergosterol in the nano-formulation can precisely act on this signaling pathway, thereby protecting neuronal cells, reducing neuronal cell death caused by ischemic stroke, maintaining intracellular homeostasis, and protecting the normal function of neuronal cells. The ergosterol can also regulate the expression and signal transduction of vascular endothelial growth factor (VEGF), promoting the proliferation, migration, and lumen formation of vascular endothelial cells, which is beneficial for angiogenesis. Furthermore, the ergosterol contributes to providing a cell source for neuroregeneration, facilitating the re-establishment of connections between damaged neuronal cells and the restoration of neurological function. The prior art lacks drugs or treatment methods that enable such precise regulation of this pathway. Based on the new discovery of the mechanism of action of ergosterol and the innovative design of the nano-formulation, this solution provides a completely new mechanistic approach for the treatment of ischemic stroke. Unlike the relatively singular treatment mechanisms in the prior art, which are primarily based on methods such as thrombolysis or surgery, this solution exerts comprehensive effects from multiple aspects including cell signaling pathway regulation, neuroprotection, angiogenesis, and neuroregeneration, demonstrating higher innovativeness and potential therapeutic value.

The nano-formulation, specifically the ergosterol-targeted micelles prepared by encapsulating the active drug ergosterol with the nanomaterial DSPE-PEG-pinacol boronate, possesses ROS-responsive properties. At the site of ischemic stroke, due to the presence of excessive ROS, the nano-formulation can rapidly respond and release ergosterol. The released ergosterol timely activates the PI3K/AKT/mTOR pathway. This synergistic effect makes the treatment more precise and effective. In contrast, drugs or formulations in the prior art often lack such a responsive release mechanism adapted to the pathological environment, making it difficult for these drugs or formulations to act at the appropriate time and location. Furthermore, characteristics of the nano-formulation such as particle size and zeta potential are conducive to its interaction with cell membranes, promoting cellular uptake of ergosterol. After the increased cellular uptake, ergosterol can more effectively enter cells to activate the PI3K/AKT/mTOR pathway. This provides more opportunities for ergosterol to function intracellularly and activate the pathway, resulting in higher cellular uptake efficiency compared to conventional drugs in the prior art.

Bioinformatics analysis revealed that the PI3K/AKT/mTOR signaling pathway plays an important role in the progression of IS. The PI3K/AKT/mTOR signaling pathway plays a significant role in central nervous system injury. Upon external stimulation, PI3K is activated to produce phosphatidylinositol (3,4,5)-trisphosphate (PIP3), which recruits and activates AKT. AKT phosphorylates downstream proteins, such as inhibiting Bad protein and caspase-9, preventing the release of cytochrome C, and thereby inhibiting apoptosis induced by ischemia. This pathway can also regulate ion channels and transport proteins on the cell membrane, thereby contributing to maintaining intracellular homeostasis, protecting the normal function of neuronal cells, and avoiding phenomena such as intracellular calcium overload caused by ischemia. Additionally, this pathway can activate endothelial nitric oxide synthase (eNOS) to produce nitric oxide (NO), thereby promoting endothelial cell migration and proliferation, and can also regulate the expression and signal transduction of VEGF, promoting the proliferation, migration, and lumen formation of vascular endothelial cells, which is beneficial for angiogenesis. The PI3K/AKT/mTOR pathway can stimulate the proliferation and differentiation of neural stem cells, guiding the neural stem cells to differentiate into neurons, thus providing a cell source for neuroregeneration. This facilitates damaged neuronal cells to re-establish connections and restore neurological function.

Compared with the prior art, the present disclosure has the following beneficial effects:
1. The nanomaterial DSPE-PEG-pinacol boronate prepared in this solution and the ergosterol-loaded brain-targeting nano-formulation, exhibit a regular spherical or quasi-spherical morphology, good dispersibility, and a uniform particle size distribution. These characteristics facilitate their circulation and distribution in vivo, allowing them to reach brain lesion sites more readily. Furthermore, the ergosterol-loaded brain-targeting nano-formulation possesses a relatively high absolute value of zeta potential, which effectively enhances the stability of the nano-formulation. This enhanced stability enables the nano-formulation to maintain its structural integrity during storage and under in vivo conditions, reducing its tendency to aggregate or decompose, and thereby contributing to improved drug stability.
2. The nano-formulation exhibits ROS-responsive properties. In a simulated ischemic stroke environment, the ergosterol-loaded nano-formulation demonstrates a higher cumulative release rate and maintains an elevated release level. This indicates that during an ischemic stroke event, locally generated ROS can trigger the rapid decomposition of the nano-formulation, resulting in drug release. This mechanism enables targeted drug release, increasing the drug concentration at the lesion site, enhancing the therapeutic efficacy, and reducing toxic side effects on normal tissues simultaneously.
3. It was first discovered in this solution that ergosterol and the nano-formulation thereof can exert a therapeutic effect against ischemic stroke by activating the PI3K/AKT/mTOR signaling pathway. Activation of this pathway leads to improved regulation of intracellular signal transduction, inhibition of cell apoptosis, and promotion of cell survival and functional recovery, thereby mediating the therapeutic action against ischemic stroke.
4. This solution is expected to effectively address the problems associated with conventional formulations, such as poor targeting, low bioavailability, and significant adverse effects upon administration. This leads to an enhanced therapeutic effect of ergosterol on ischemic stroke, demonstrating superior efficacy, safety, and application potential in the treatment of ischemic stroke. Furthermore, it is expected to improve patient treatment outcomes and prognosis, and enhance patients'quality of life. This solution provides a novel approach and method for treating ischemic stroke, establishes a new therapeutic direction based on nano-formulations and the regulation of specific signaling pathways, and offers valuable references for subsequent related research and drug development.

### BRIEF DESCRIPTION OF THE DRAWINGS

To illustrate the technical solutions in the embodiments of the present disclosure or in the prior art more clearly, the following provides a brief introduction to the accompanying drawings required for describing the embodiments or the prior art. Obviously, the accompanying drawings in the following description show only some embodiments of the present disclosure. Persons of ordinary skill in the art may obtain other drawings based on these accompanying drawings without creative effort.

FIG. 1 shows the synthetic route for the nanomaterial DSPE-PEG-pinacol boronate in Example 1;

FIG. 2 shows the nuclear magnetic resonance (NMR) hydrogen-1 spectrum (¹H NMR) of the nanomaterial DSPE-PEG-pinacol boronate (A of FIG. 2), the NMR carbon-13 spectrum (¹³C NMR) of DSPE-PEG-pinacol boronate (B of FIG. 2), the ultraviolet (UV) spectrum of DSPE-PEG-OH/pinacol boronate (C of FIG. 2), and the infrared (IR) spectrum of DSPE-PEG-OH/pinacol boronate (D of FIG. 2) in Experimental Example 1;

FIG. 3 shows the dynamic light scattering (DLS) particle size results for the nanomaterial DSPE-PEG-pinacol boronate in Experimental Example 1;

FIG. 4 shows transmission electron microscopy (TEM) images of a blank nano-formulation, a drug ergosterol-loaded nano-formulation, and a hydrogen peroxide (H₂O₂)-treated ergosterol-loaded nano-formulation in Experimental Example 2; A of FIG. 4 shows the blank nano-formulation, B of FIG. 9 shows the drug ergosterol-loaded nano-formulation, and C of FIG. 9 shows the H₂O₂-treated ergosterol-loaded nano-formulation;

FIG. 5 shows particle size detection results for a blank nano-formulation formed from the nanomaterial, a drug ergosterol-loaded nano-formulation, and an H₂O₂-treated ergosterol-loaded nano-formulation in Experimental Example 2; A of FIG. 5 shows the blank nano-formulation, B of FIG. 5 shows the drug ergosterol-loaded nano-formulation, and C of FIG. 5 shows the H₂O₂-treated ergosterol-loaded nano-formulation;

FIG. 6 shows cellular uptake detection results for the ergosterol-loaded nano-formulation prepared in Experimental Example 2;

FIG. 7 shows particle size, zeta potential detection, and encapsulation efficiency of the nano-formulation in Experimental Example 2; A of FIG. 7 shows particle size detection results for the ergosterol-loaded nano-formulation at different time points; A of FIG. 7 shows zeta potential detection results for the ergosterol-loaded nano-formulation at different time points; and C of FIG. 7 shows encapsulation efficiency detection results for the ergosterol-loaded nano-formulation at different time points;

FIG. 8 shows drug release results for ergosterol drug and the ergosterol-loaded nano-formulation in a pH 7.4 medium and a pH 7.4+100 μM H₂O₂ medium, respectively, in Experimental Example 2; A of FIG. 8 shows drug release results in the pH 7.4 medium, and B of FIG. 8 shows drug release results in the pH 7.4 +100 μM H₂O₂ medium;

FIG. 9 shows an image of a rat with an established ischemic stroke model in Experimental Example 3 (A of FIG. 9), and the effect of different treatments on the Longa score of rats (B of FIG. 9);

FIG. 10 shows rat brain tissue, cerebral infarction volume, and hematoxylin-eosin (H&E) staining images in Experimental Example 3; A of FIG. 10 shows 2,3,5-triphenyltetrazolium chloride (TTC) staining results of rat brain tissue in different treatment groups; B of FIG. 10 shows cerebral infarction rates in rats of different treatment groups; and C of FIG. 10 shows H&E staining results showing the morphological structure of rat brain tissue in different treatment groups;

FIG. 11 shows the effects of ergosterol and the nano-formulation thereof on the expression of related proteins in Experimental Example 3: a comparative study based on a sham surgery group and a model group; A of FIG. 11 shows Western blot detection of related protein expression levels; B of FIG. 11 shows a quantitative graph of p-mTOR/mTOR protein expression; C of FIG. 11 shows a quantitative graph of p-AKT/AKT protein expression; and D of FIG. 11 shows a quantitative graph of p-PI3K/PI3K protein expression;

FIG. 12 shows in vivo imaging results of rats at 1 hour and 4 hours after administration of the ergosterol nano-formulation prepared in Comparative Example 1 (A of FIG. 12, B of FIG. 12) and the ergosterol-loaded nano-formulation prepared in Example 2 (C of FIG. 12, D of FIG. 12).

### DETAILED DESCRIPTION OF THE EMBODIMENTS

To make the technical problems to be solved, the technical solutions, and the advantages of the present disclosure clearer, the following describes the present disclosure in detail with reference to the accompanying drawings and specific examples.

The following examples are provided to illustrate the present disclosure but are not intended to limit the scope of the present disclosure. Modifications or substitutions made to the methods, steps, or conditions of the present disclosure without departing from the spirit and essence of the present disclosure fall within the scope of the present disclosure.

Unless otherwise specified, the technical means used in the examples are conventional means well-known to those skilled in the art; unless otherwise specified, the reagents used in the examples are commercially available.

### Example 1: Preparation of the Nanomaterial DSPE-PEG-Pinacol Boronate

S1. Activation of pinacol boronate: 1.8 g of 4-(hydroxymethyl)phenylboronic acid pinacol ester (PBAP) and 2.2 g of N,N′-carbonyldiimidazole (CDI) were precisely weighed and placed in a 50 mL two-necked flask; 20 mL of anhydrous dichloromethane (DCM) was added to the flask; after the solids dissolved, the mixture was reacted on a constant-temperature magnetic stirrer for one hour; subsequently, the reaction mixture was extracted with a double volume of DCM, and the organic phase was washed three times with deionized water; the organic phase was further extracted with a saturated sodium chloride (NaCl) solution and finally dried with an appropriate amount of anhydrous sodium sulfate to obtain the activated pinacol boronate, CDI-PBAP.
S2. Preparation of the nanomaterial DSPE-PEG-pinacol boronate: 1.6 g of the CDI-PBAP obtained in step S1, 0.35 g of β-cyclodextrin (B-CD), 0.9 g of 4-dimethylaminopyridine (DMAP), and 0.4 g of DSPE-PEG were added into dimethyl sulfoxide (DMSO) to react; the reaction solution was precipitated by adding anhydrous diethyl ether, and the precipitate was washed three times with anhydrous diethyl ether; the precipitate was collected by centrifugation and freeze-dried under vacuum for approximately 24 hours to obtain the nanomaterial DSPE-PEG-pinacol boronate.

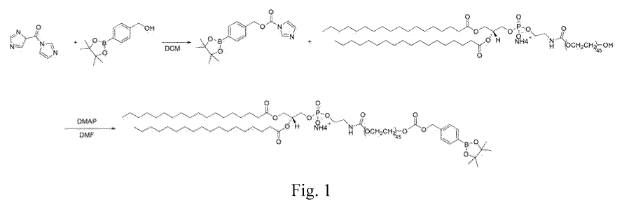

The synthetic route for the nanomaterial DSPE-PEG-pinacol boronate is shown in FIG. 1.

### Example 2: Preparation of the Ergosterol-Loaded Brain-Targeting Nano-Formulation

S1. 5 mg of ergosterol and 40 mg of the DSPE-PEG-pinacol boronate prepared in Example 1 were weighed and placed in a round-bottom flask; an appropriate amount of chloroform was added to the flask, and the mixture was stirred using a magnetic stirrer at room temperature until the materials and the drug were completely dissolved; the round-bottom flask was connected to a rotary evaporator, and slow rotary evaporation was performed at 40° C. and 0.1 MPa until the organic solvent was completely evaporated, thereby forming a uniform, thin, and transparent lipid film on the inner wall of the flask.
S2. An appropriate amount of PBS buffer was slowly added to the flask containing the formed lipid film; the mixture was gently stirred under a 37° C. water bath condition to allow complete hydration of the lipid film; subsequently, the resulting suspension was subjected to ultrasonication and dialysis purification, followed by freeze-drying to obtain the ergosterol-loaded brain-targeting nano-formulation.

### Comparative Example 1: Preparation of the Ergosterol-Loaded Nano-Formulation Without Pinacol Boronate

S1. Preparation of the nanomaterial DSPE-PEG: 0.35 g of B-CD, 0.9 g of DMAP, and 0.4 g of DSPE-PEG were added into DMSO to react; the reaction solution was precipitated by adding anhydrous diethyl ether, and the precipitate was washed three times with anhydrous diethyl ether; the precipitate was collected by centrifugation and freeze-dried under vacuum for approximately 24 hours to obtain the nanomaterial DSPE-PEG.
S2. 5 mg of ergosterol and 40 mg of the DSPE-PEG prepared in step S1 of this Comparative Example 1 were weighed and placed in a round-bottom flask; an appropriate amount of chloroform was added to the flask, and the mixture was stirred using a magnetic stirrer at room temperature until the materials and the drug were completely dissolved; the round-bottom flask was connected to a rotary evaporator, and slow rotary evaporation was performed until the organic solvent was completely evaporated, thereby forming a uniform, thin, and transparent lipid film on the inner wall of the flask.
S3. An appropriate amount of PBS buffer was slowly added to the flask containing the formed lipid film; the mixture was gently stirred under a 37° C. water bath condition to allow complete hydration of the lipid film; subsequently, the resulting suspension was subjected to ultrasonication and dialysis purification, followed by freeze-drying to obtain the ergosterol-loaded nano-formulation without pinacol boronate.

### Experimental Example 1: Investigation of the Characterization of the Nanomaterial DSPE-PEG-Pinacol Boronate

(1) Characterization by ¹H NMR, ¹³C NMR, UV, and IR Spectroscopy

An appropriate amount of the nanomaterial DSPE-PEG-pinacol boronate prepared in Example 1 was taken and dissolved in deuterated dimethyl sulfoxide to a concentration of 1-10 mM. The solution was transferred into an NMR tube, and the NMR tube was placed into a nuclear magnetic resonance spectrometer for ¹H NMR and ¹³C NMR measurements.

The nanomaterial DSPE-PEG-pinacol boronate was ground into a fine powder and then uniformly mixed with dried potassium bromide (KBr) powder in a specific ratio. The mixture was placed into a pellet die, and an appropriate pressure was applied using a pellet press for several minutes to form a transparent pellet suitable for IR detection.

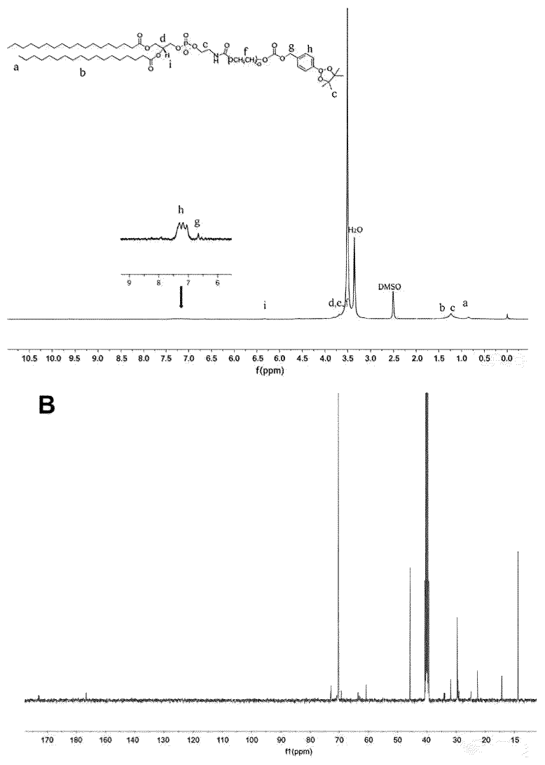

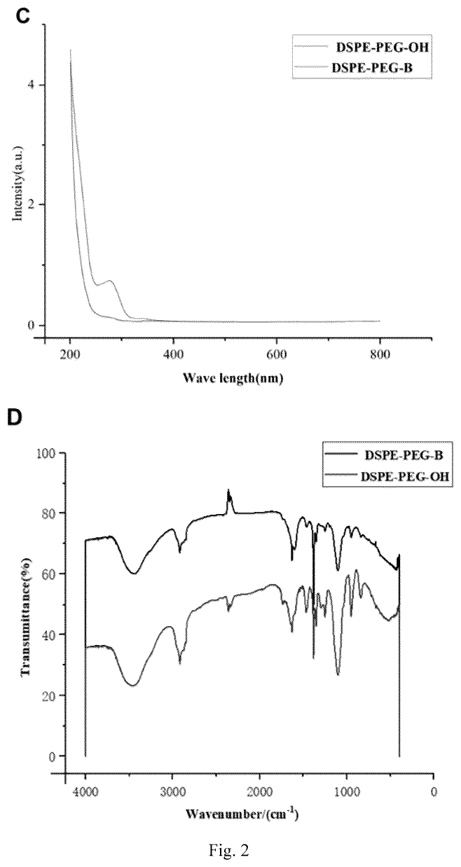

The NMR detection results are shown in FIG. 2A and FIG. 2B, the UV spectrum is shown in FIG. 2C, and the IR detection results are shown in FIG. 2D. Based on these detection results, the successful preparation of the nanomaterial DSPE-PEG-pinacol boronate is confirmed.

### (2) Particle Size

An appropriate amount of the nanomaterial DSPE-PEG-pinacol boronate was taken and diluted with ultrapure water to a suitable concentration. The diluted sample was added to a sample cell for particle size detection of the prepared nanomaterial DSPE-PEG-pinacol boronate. The detection results, shown in FIG. 3, indicate an average particle size of 26.92 nm and a polydispersity index (PDI) of 0.234 for the prepared nanomaterial, demonstrating a uniform particle size distribution.

### Experimental Example 2: Investigation of the Basic Characterization of the Ergosterol-Loaded Brain-Targeting Nano-Formulation

### (1) Morphological Characterization

A TEM-specific copper grid was subjected to hydrophilic treatment. Approximately 250 μg/mL solutions of the blank material DSPE-PEG-pinacol boronate, the ergosterol-loaded nano-formulation prepared in Example 2, and the H₂O₂-treated ergosterol-loaded nano-formulation were separately dropped onto the front side of the copper grid. After standing for 2 minutes, an appropriate amount of phosphotungstic acid stain was dropped onto the copper grid, and the liquid on the copper grid was absorbed with filter paper. Phosphotungstic acid stain was dropped again, and after standing for another 2 minutes, the liquid was absorbed with filter paper. After repeating the phosphotungstic acid staining once more, the copper grid was dried under an incandescent lamp. The copper grid was then placed into a TEM to examine the morphology of the samples.

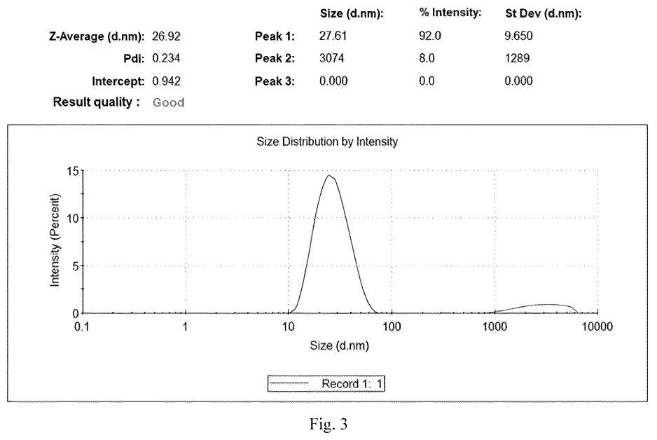

The detection results are shown in FIG. 4. The nanomaterial DSPE-PEG-pinacol boronate and the ergosterol-loaded nano-formulation exhibited a regular spherical or quasi-spherical morphology with favorable dispersibility. After loading with ergosterol, the structure of the nanomaterial did not undergo significant changes, indicating that the nanomaterial possesses a good encapsulation capacity for ergosterol. Compared to the aforementioned two, the H₂O₂-treated ergosterol-loaded nano-formulation showed obvious changes: the particle distribution was no longer uniform, and changes in the morphology of some particles could be observed, showing signs of decomposition. This provides intuitive evidence for studying the drug release mechanism and the stability of the nano-formulation.

### (2) Determination of Particle Size, Zeta Potential, and PDI

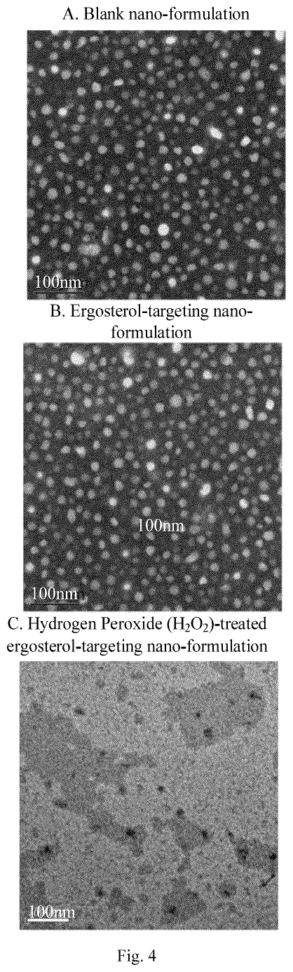

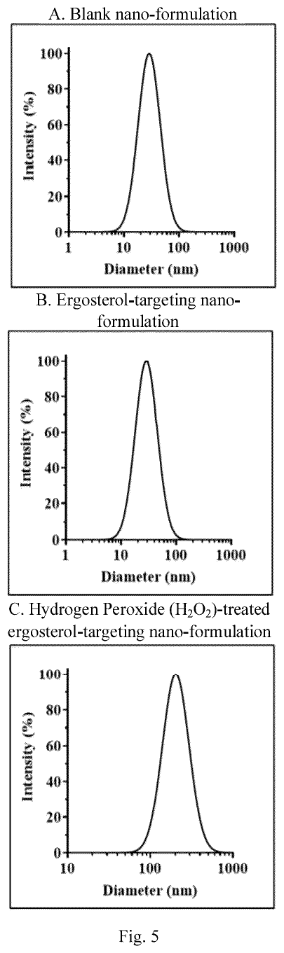

A dynamic light scattering (DLS) particle size analyzer was used to characterize the particle size, zeta potential, and PDI of the blank material DSPE-PEG-pinacol boronate, the ergosterol-loaded nano-formulation, and the H₂O₂-treated ergosterol-loaded nano-formulation. Determination of Particle Size and PDI: After preheating the instrument for 30 minutes, the quartz cuvette was rinsed 2-3 times with purified water. The nano-formulation was diluted with purified water at a certain ratio and then added to the cuvette. The instrument was set to perform 3 measurement cycles per sample with a defined single-cycle duration, and “Water” was selected as the test solvent. After the instrument stabilized, measurement was initiated. The particle size test results are shown in FIG. 5. The PDI result for the ergosterol-loaded nano-formulation was 0.196±0.002. Based on the characterization results, the particle size of the nanomaterial DSPE-PEG-pinacol boronate was uniformly distributed around 30 nm, with no significant change observed before and after drug loading. However, the particle size of the H₂O₂-treated ergosterol-loaded nano-formulation increased significantly. This indicates that H₂O₂ treatment can simulate the rapid decomposition of the targeting nano-formulation induced in an ischemic stroke environment, leading to drug release, thereby potentially enhancing the therapeutic effect on ischemic stroke and reducing toxic side effects on normal tissues.

Determination of Zeta Potential: After preheating the instrument for 30 minutes, the quartz cuvette was rinsed 2-3 times with purified water. The ergosterol nano-formulation was diluted with purified water at a certain ratio and then added to the cuvette. The electrode plate was inserted into the cuvette and connected to the instrument. The cycle time and number of cycles were set, and “Water” was selected as the test solvent. After the instrument stabilized, measurement was initiated. The measured zeta potential of the ergosterol-loaded nano-formulation was −23.88±0.38 mV. The high absolute value of the zeta potential effectively increases the stability of the ergosterol-loaded nano-formulation, enhances the transmembrane activity of the drug, and thereby promotes an improvement in the bioavailability of the drug.

### (3) Cellular Uptake Experiment

Cy2-labeled ergosterol (excitation wavelength around 480 nm, emission wavelength at 508 nm) was used to prepare the Cy2-labeled ergosterol-loaded nano-formulation according to the method described above. PC12 cells in the logarithmic growth phase were seeded onto a 6-well plate (1×10⁵ cells/mL, 2 mL per well). After the cells adhered, the culture medium was discarded. Two formulations with a labeling concentration of 20 nM were incubated with HCT 116 cells in a confocal culture dish. The cells were cultured for 2, 4, and 12 hours. The cells were washed three times with pre-cooled PBS and then incubated with DAPI (5 μg/mL) to facilitate observation under a fluorescence microscope.

The detection results are shown in FIG. 6. After 1 hour of incubation, a small amount of green fluorescence could be detected around the cell nuclei. As the incubation time extended to 2, 4, and 6 hours, the uptake of the Cy 2-labeled ergosterol-loaded nano-formulation by PC12 cells increased, and the fluorescence intensity enhanced. This indicates that the Cy2-labeled ergosterol-loaded nano-formulation can be effectively taken up by cells, thereby exerting a therapeutic effect.

### (4) Stability Testing of the Ergosterol-Loaded Nano-Formulation

The stability of the ergosterol-loaded nano-formulation at different storage times was investigated. Samples were stored in sealed vials under conditions of 4° C. for 30 days. Samples were taken at corresponding times (1, 15, and 30 days) to determine the particle size distribution, zeta potential, and encapsulation efficiency to evaluate the storage stability of the ergosterol-loaded nano-formulation.

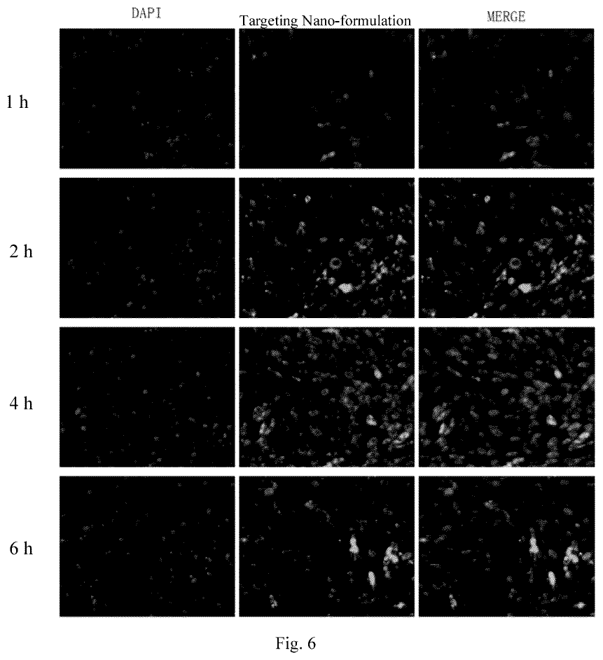

The determination results are shown in FIG. 7. After a preliminary 30-day stability test, although the values for particle size distribution, encapsulation efficiency, and zeta potential of the ergosterol-loaded nano-formulation all increased within 30 days, with drug loading ranging from 8.05% to 18.33% and encapsulation efficiency from 85.20% to 93.77%, there were no statistically significant changes (P>0.05). This indicates that the nano-formulation possesses good stability and can be stably stored under the aforementioned conditions.
(5) In Vitro Release of the Nano-Formulation

The in vitro release of ergosterol and the ergosterol-loaded nano-formulation was determined using the dialysis method. Ergosterol and the ergosterol-loaded nano-formulation were separately placed into dialysis devices with a molecular weight cut-off of 100 kDa. The buffer media were pH 7.4 PBS and pH 7.4 PBS+100 μM H₂O₂, respectively. At time points of 15, 30, 45, 60, 90, 120, 180, 240, 360, 480, 600, 720, 1440, 2160, and 2880 minutes, 1 mL of the release medium was taken and replaced with an equal volume of the corresponding fresh medium. High-performance liquid chromatography (HPLC) was used to detect the content of released ergosterol.

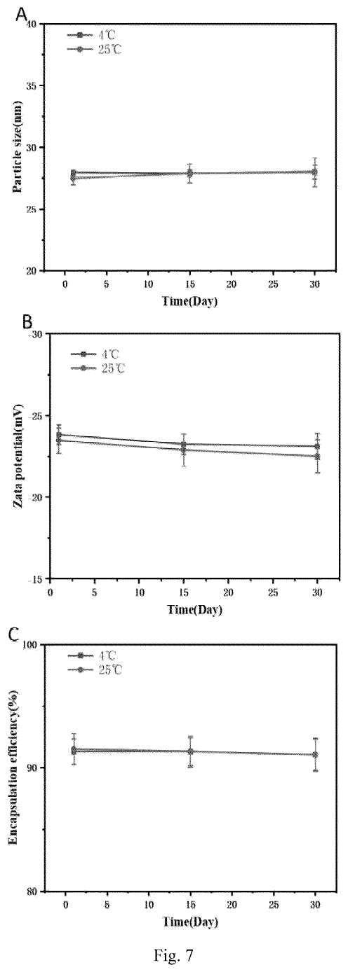

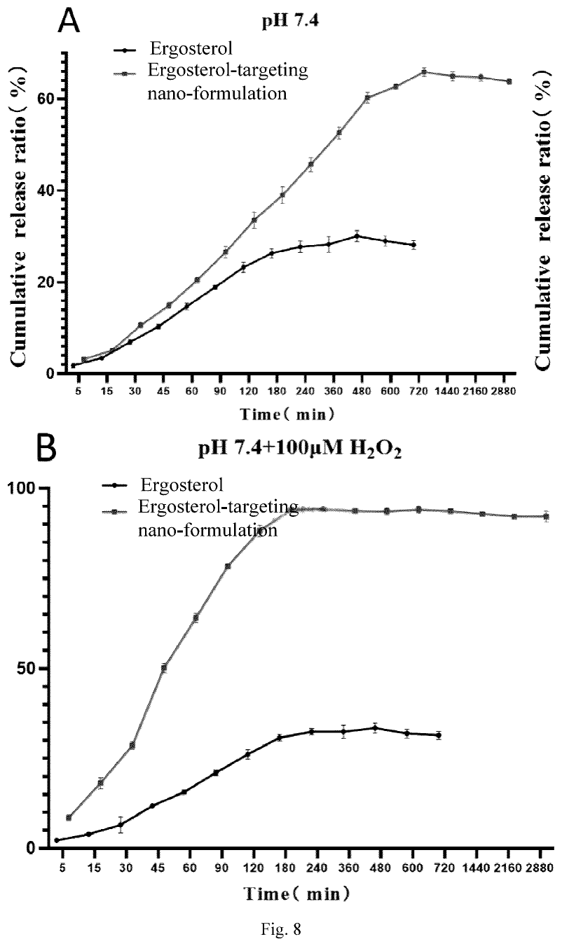

The results are shown in FIG. 8. In the pH 7.4 medium, the cumulative release rate of the ergosterol-loaded nano-formulation was significantly higher than that of ergosterol alone. In an environment of pH 7.4 containing 100 μM H₂O₂, the cumulative release rate of the ergosterol-loaded nano-formulation was even higher and maintained a relatively high release level throughout the process, while the cumulative release rate of ergosterol itself was relatively low. This indicates that formulating ergosterol into the ergosterol-loaded nano-formulation can effectively improve drug stability and reduce the impact of external environmental factors. H₂O₂ treatment can simulate the rapid decomposition of the nano-formulation induced in an ischemic stroke environment, leading to drug release.

### Experimental Example 3: Investigation of the Use of the Ergosterol-Loaded Nano-Formulation in Treating Ischemic Stroke

Construction of a Rat Model of Ischemic Stroke: Sprague Dawley (SD) rats, weighing 300g±10g and aged 11-12 weeks, were used. The rats were anesthetized with chloral hydrate, placed in a supine position, and fixed on a rat board. The right common carotid artery (CCA), internal carotid artery (ICA), and external carotid artery (ECA) were bluntly separated using ophthalmic forceps and prepared with suture threads for later ligation. The proximal ends of the ECA and CCA were ligated, and the ICA was temporarily clamped with an arterial clip. The CCA was lifted with ophthalmic forceps, and a small incision was made. A prepared suture thread was quickly inserted along the CCA towards the ICA. Insertion was stopped when slight resistance was felt at the tip of the thread. The prepared ligature on the CCA was then tightened to prevent movement or dislodgement of the thread and to control bleeding. The prepared ligature was tied, and the occlusion time was recorded. After 1 hour of ischemia, the suture thread was removed to allow reperfusion. The surgical procedure for the sham-operated group was identical to the above, except that the suture thread was inserted only 15 mm in depth, without occluding blood flow in the middle cerebral artery. Successful modeling was indicated when the rat was lifted by the tail and exhibited left forelimb flexion and adduction, along with a typical circling behavior when moving.

### 1. Effects of Ergosterol and the Ergosterol-Loaded Nano-Formulation on Neurological Function and Pathological Status in Rats

(1) The Longa scoring method was employed to quantify the degree of neurological deficit in animals through a series of behavioral observations. The score ranges from 0 to 4, where: 0 indicates no neurological deficit; 1 indicates failure to fully extend the forepaw on the paralyzed side; 2 indicates circling towards the paralyzed side when walking; 3 indicates falling towards the paralyzed side when walking; 4 indicates inability to walk spontaneously, with loss of consciousness. A higher score indicates worse neurological behavioral status and more severe neurological deficit. In the experiment, the Longa score was used to preliminarily assess the success of the model. Animals with scores between 1 and 3 were generally included in the next step of the experiment, while animals with scores of 4 or 5 were excluded, as this might indicate the animal's condition was unsuitable for the experiment. Rats in each group were scored before treatment and on days 1, 3, 7, 14, and 28 after treatment.

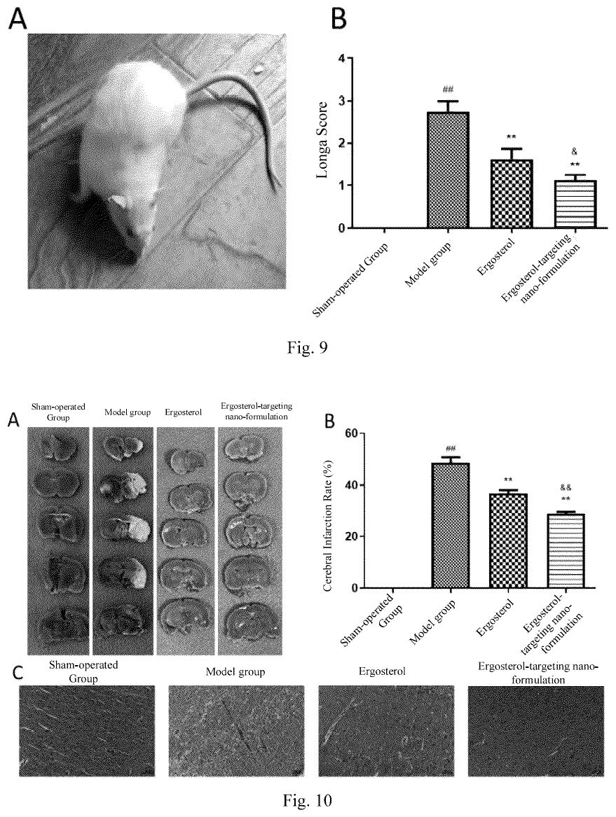

The determination results are shown in FIG. 9. The model group had the highest Longa score, indicating severe neurological deficit in the model group rats. Compared to the model group, the ergosterol group showed a significant decrease in score (P<0.01), indicating that ergosterol could improve the neurological deficit caused by ischemic stroke to a certain extent. Compared to the ergosterol group, the ergosterol-loaded nano-formulation group prepared according to the present disclosure showed a significantly lower score (P<0.01), indicating that the ergosterol-loaded nano-formulation could markedly and effectively improve the neurological deficit caused by ischemic stroke and was more effective than ergosterol alone.
(2) The macroscopic appearance of the rat brain is one of the important indicators for assessing the severity of brain damage. At the experimental endpoint, the rats were euthanized, and the brains were separated, dried with filter paper, and photographed. The results (FIG. 10A) showed that the brain tissue of the model group had an uneven color, with some white or pale areas representing cerebral infarction sites and ischemic damage, indicating successful induction of brain injury in the model group. Both ergosterol and the ergosterol-loaded nano-formulation reduced the ischemic area to varying degrees, suggesting a protective effect, with the ergosterol-loaded nano-formulation showing the most significant protective effect, closest to that of the sham-operated group. Based on the cerebral infarction volume measurement results (FIG. 10B), the model group had the largest cerebral infarction volume, indicating severe brain damage in the model group rats. Compared to the model group, the ergosterol group showed a significant decrease in cerebral infarction volume (P<0.01), indicating that ergosterol had a certain improving effect on the cerebral infarction caused by ischemic stroke. Compared to the ergosterol group, the ergosterol-loaded nano-formulation group prepared according to the present disclosure showed a significantly lower cerebral infarction volume (P<0.01), indicating that the ergosterol-loaded nano-formulation could markedly improve the cerebral infarction caused by ischemic stroke.
(3) H&E staining results of brain tissue directly reflected the brain damage. As seen in FIG. 10C, the tissue sections from the sham-operated group showed a relatively normal tissue structure, with relatively orderly cell arrangement, uniform intercellular spaces, and no obvious signs of inflammation or damage. The model group showed obvious tissue damage: disordered cell arrangement, enlarged intercellular spaces, infiltration of a considerable number of inflammatory cells, and the presence of some voids or necrotic areas in the tissue. The ergosterol group showed a certain degree of tissue repair. Compared to the model group, the cell arrangement was more orderly, inflammatory cell infiltration was reduced, and necrotic areas were decreased. The ergosterol-loaded nano-formulation group showed the best tissue repair effect: the tissue structure was near-normal, cell arrangement was orderly, inflammatory cell infiltration was significantly reduced, and necrotic areas were minimal. This indicates that, compared to ergosterol, the ergosterol-loaded nano-formulation prepared according to the present disclosure has a more significant effect in treating ischemic stroke.

### 2. Protein Immunoblotting (Western Blot) Assay

Through the Western Blot assay, the expression levels of proteins including p-PI3K, p-AKT, p-mTOR, mTOR, PI3K, and AKT in rat brain tissue were measured, further verifying that ergosterol and the formulation thereof exert therapeutic effects on ischemic stroke by activating the PI3K/AKT/mTOR pathway.
(1) Sample Preparation: After the animal experiments, rat brain tissue samples were collected. RIPA lysis buffer was added, and the samples were placed on ice for 50 minutes for thorough lysis. The lysate was pipetted and transferred to 1.5 mL centrifuge tubes and centrifuged at 4° C., 12,000 rpm for 20 minutes, and the supernatant was collected. The total protein concentration of each sample was determined using the bicinchoninic acid (BCA) method, and the samples were diluted to the same concentration using RIPA lysis buffer. An appropriate amount of 5× Loading Buffer was added to each sample volume, mixed well, and then denatured continuously in boiling water for 15 minutes. After brief vortexing and centrifugation, the samples could be used directly for detection or stored at −20° C.
(2) The Western Blot assay was performed to measure changes in p-PI3K, p-AKT, p-mTOR, mTOR, PI3K, and AKT proteins in rat brain tissue.

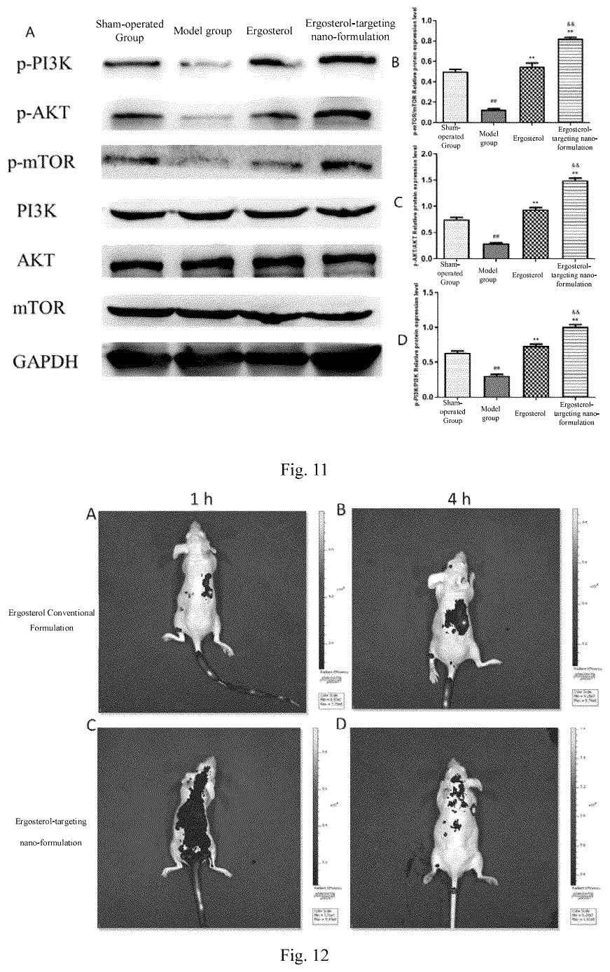

The results are shown in FIG. 11. Compared to the sham-operated group, the expression levels of p-PI3K, p-AKT, p-mTOR, mTOR, PI3K, and AKT proteins in the model group were significantly decreased (P<0.01). After drug treatment, both ergosterol and the ergosterol-loaded nano-formulation significantly increased the expression levels of the aforementioned proteins. Furthermore, compared to ergosterol, the increase in the expression levels of these proteins was more pronounced after treatment with the ergosterol-loaded nano-formulation (P<0.01). The Western Blot results indicate that ergosterol and the formulation thereof can exert therapeutic effects on ischemic stroke by activating the PI3K/AKT/mTOR pathway, and the ergosterol-loaded nano-formulation exhibits a better therapeutic effect.
3. In Vivo Imaging Assay in Rats

Through the in vivo imaging assay, the distribution of the drug formulations in different parts of the rat body was detected, visually demonstrating the brain-targeting advantage of the ergosterol-loaded nano-formulation prepared according to the present disclosure for treating ischemic stroke.

The results are shown in FIG. 12. Two groups of rats were intravenously injected via the tail vein with the pinacol boronate-free nano-reagent prepared in Comparative Example 1 and the ergosterol-loaded nano-reagent prepared in Example 2, respectively. It was clearly visible that the nano-reagent prepared in Comparative Example 1, whether at 1 hour or 4 hours after tail vein injection, was mainly concentrated in the rat's tail and lower abdomen, showing no targeting tendency towards the brain (FIG. 12A, FIG. 12B). In contrast, the ergosterol-loaded nano-reagent prepared according to the present disclosure showed red fluorescence mainly concentrated in the rat's tail and lower abdominal regions within 1 hour after injection, indicating that the drug was primarily located in these areas shortly after injection (FIG. 12C). At 4 hours after tail vein injection of the ergosterol-loaded nano-formulation, the red fluorescence had obviously moved and accumulated in the brain region of the rat (FIG. 12D). This result visually demonstrates that the ergosterol-loaded nano-reagent prepared according to the present disclosure possesses good brain-targeting properties, i.e., the ability to effectively deliver the drug to the brain, providing a unique advantage in treating ischemic stroke.

In summary, the present disclosure successfully constructed a novel ergosterol-loaded nano-formulation for targeted treatment of ischemic stroke. Firstly, a novel nanomaterial DSPE-PEG-pinacol boronate was successfully synthesized. The present disclosure provides this nano-formulation, preparation method thereof and use. This nano-formulation can effectively address problems such as poor drug targeting and low bioavailability, opens up new treatment methods for ischemic stroke, and enhances the targeted therapeutic capability of the drug. Concurrently, it was discovered that ergosterol exerts a therapeutic effect on ischemic stroke by activating the PI3K/AKT/mTOR pathway. The above findings provide a new direction for the treatment of ischemic stroke.

The foregoing describes only preferred embodiments of the present disclosure, and the scope of protection of the present disclosure is not limited to the above examples. Improvements and modifications obtained by those skilled in the art without departing from the technical concept of the present disclosure should also be considered within the protection scope of the present disclosure.

What is claimed is:

1. an ergosterol-loaded brain-targeting nano-formulation, wherein the nano-formulation comprises ergosterol, pinacol boronate and DSPE-PEG; wherein the pinacol boronate modifies DSPE-PEG to form a DSPE-PEG-pinacol boronate nanomaterial for encapsulating the ergosterol; and
a method for preparing the DSPE-PEG-pinacol boronate nanomaterial comprises the following steps:
S1, activating pinacol boronate: mixing 4-(hydroxymethyl)phenylboronic acid pinacol ester with N,N′-carbonyldiimidazole, adding anhydrous dichloromethane, and stirring to react; after the reaction is completed, extracting with anhydrous dichloromethane, washing three times with deionized water, further extracting the resulting organic phase with a saturated sodium chloride solution, and drying over anhydrous sodium sulfate to obtain the activated pinacol boronate;
wherein the mass ratio of the 4-(hydroxymethyl)phenylboronic acid pinacol ester to the N,N′-carbonyldiimidazole is (1-2):(1-2.5), and the stirring time is 1 h; and
S2, preparing the DSPE-PEG-pinacol boronate nanomaterial: dissolving the activated pinacol boronate obtained in S1, β-cyclodextrin, 4-dimethylaminopyridine, and DSPE-PEG together in dimethyl sulfoxide to react; adding anhydrous diethyl ether to the reaction solution for precipitation, washing with anhydrous diethyl ether, centrifuging to collect a precipitate, and performing freeze-drying under a vacuum environment to obtain the DSPE-PEG-pinacol boronate nanomaterial; wherein the mass ratio of the activated pinacol boronate to the β-cyclodextrin to the 4-dimethylaminopyridine is (1-2):(0.1-1):(0.5-1).

2. The ergosterol-loaded brain-targeting nano-formulation according to claim 1, wherein a zeta potential of the nano-formulation is −23.88±0.38 mV, a particle size of the nano-formulation is approximately 30 nm, a drug loading capacity of the nano-formulation is 8.05% to 18.33%, and an encapsulation efficiency of the nano-formulation is 85.20% to 93.77%.

3. A method for preparing the ergosterol-loaded brain-targeting nano-formulation according to any one of claims 1 to 2, comprising the following steps:
S1, activating pinacol boronate: mixing 4-(hydroxymethyl)phenylboronic acid pinacol ester with N,N′-carbonyldiimidazole, adding anhydrous dichloromethane, and stirring to react; after the reaction is completed, extracting with anhydrous dichloromethane, washing three times with deionized water, further extracting the resulting organic phase with a saturated sodium chloride solution, and drying over anhydrous sodium sulfate to obtain the activated pinacol boronate; wherein the mass ratio of the 4-(hydroxymethyl)phenylboronic acid pinacol ester to the N,N′-carbonyldiimidazole is (1-2):(1-2.5), and the stirring time is 1 h;
S2, preparing the DSPE-PEG-pinacol boronate nanomaterial: dissolving the activated pinacol boronate obtained in S1, β-cyclodextrin, 4-dimethylaminopyridine, and DSPE-PEG together in dimethyl sulfoxide to react; adding anhydrous diethyl ether to the reaction solution for precipitation, washing with anhydrous diethyl ether, centrifuging to collect a precipitate, and performing freeze-drying under a vacuum environment to obtain the DSPE-PEG-pinacol boronate nanomaterial;
wherein the mass ratio of the activated pinacol boronate to the β-cyclodextrin to the 4-dimethylaminopyridine is (1-2):(0.1-1):(0.5-1); and
S3, preparing the nano-formulation: placing ergosterol and the DSPE-PEG-pinacol boronate nanomaterial obtained in S2 in a round-bottom flask, wherein the mass ratio of the ergosterol to the DSPE-PEG-pinacol boronate nanomaterial is 1:(4-12); adding an organic solvent; stirring until complete dissolution; connecting to a rotary evaporator for slow rotary evaporation until the organic solvent is completely evaporated and a lipid film is formed on an inner wall of the round-bottom flask; adding a phosphate-buffered saline (PBS) buffer solution; stirring in a 37° C. water bath to allow the lipid film to fully hydrate to form a suspension; performing ultrasonication and dialysis purification on the suspension; and freeze-drying to obtain the nano-formulation.

4. The preparation method according to claim 3, wherein the organic solvent in step S3 is selected from any one or more of methanol, dichloromethane, and chloroform.

5. The preparation method according to claim 4, wherein a temperature for the slow rotary evaporation in step S3 is from room temperature to 45° C., and a pressure for the slow rotary evaporation in step S3 is 0.05-0.15 MPa.

6. Use of the ergosterol-loaded brain-targeting nano-formulation according to claim 1, or the ergosterol-loaded brain-targeting nano-formulation obtained by the preparation method according to any one of claims 3 to 5, in the preparation of a medicament for targeted therapy of a brain disease; wherein the brain disease is ischemic stroke.
